(19) United States
(12) Patent Application Publication
(10) Pub. No.: US 2023/0174704 A1
(43) Pub. Date: Jun. 8, 2023

(54) POLYURETHANE INSULATING FOAMS AND PRODUCTION THEREOF

(71) Applicant: EVONIK OPERATIONS GMBH, Essen (DE)

(72) Inventors: Guangyu Dai, Shanghai (CN); Michael SUCHAN, Recklinghausen (DE); Carsten SCHILLER, Ratingen (DE)

(73) Assignee: EVONIK OPERATIONS GMBH, Essen (DE)

(21) Appl. No.: 17/919,826

(22) PCT Filed: Apr. 21, 2020
(86) PCT No.: PCT/CN2020/085902

§ 371 Date: Oct. 19, 2022

(51) Int. Cl. C08G 18/08; C08G 18/12; C08G 18/48; C08G 18/61; C08G 18/18; C08G 18/22; C08J 9/14; C08J 9/00

(52) U.S. Cl. CPC C08G 18/14; C08G 18/12; C08G 18/48; C08G 18/61; C08G 18/18; C08G 18/22; C08J 9/141; C08J 9/146; C08J 9/0019; C08G 2101/00

(57) ABSTRACT

A process is described for producing PU foams, especially rigid PU foams, based on foamable reaction mixtures containing polyisocyanates, compounds having reactive hydrogen atoms, blowing agents, foam stabilizers, and possibly further additives, wherein specific perfluoropolyethers are additionally used.

The present invention is in the field of rigid polyurethane foams. More particularly, it relates to the production of rigid polyurethane foams using specific perfluoropolyethers, and additionally to the use of the foams which have been produced therewith.

The production of polyurethane foams by foaming foamable reaction mixtures based on isocyanates, compounds having reactive hydrogen atoms, blowing agents, stabilizers and possibly further additives nowadays is operated on a large industrial scale. To this end, all components except for the isocyanate are generally preformulated to give a processable mixture and this mixture is mixed with the isocyanate in a foaming installation. Foam formation and polyaddition reaction occur simultaneously in the initially liquid reaction mixture until the composition has cured to give the desired foam.

To produce thermoset insulating foams, it may for example be desirable to produce rigid foams having a preferably relatively low foam density of <60 kg/m³ and preferably a maximum number of small closed cells (high cell density). The cells should in this case preferably be distributed uniformly over the entire moulding, that is to say should not exhibit a gradient.

A blowing gas is necessary for such a foam to be able to form. This may for example be CO₂ which arises from the reaction of isocyanate with water or is additionally added and/or an added low-boiling organic liquid.

For the sake of completeness it should be mentioned that in addition to foam formation during the polymerization reaction, as described here for the polyurethane formation, the foam formation can also take place in the extrusion process. However, these extrusion processes should be distinguished in principle from the polyurethane foam processes described here. WO 2002/034823 for instance describes an extrusion process for thermoplastics that leads to the formation of multimodal thermoplastic polymer foams. In contrast, the non-thermoplastic, instead thermoset, polyurethane foam systems that are preferably considered in the present case preferably feature a generally uniform, monomodal cell size distribution and also cannot be obtained by extrusion processes.

Rigid polyurethane and polyisocyanurate foams are usually produced using cell-stabilizing additives to ensure a fine-celled, uniform and low-defect foam structure and hence to exert an essentially positive influence on the performance characteristics, particularly the thermal insulation capacity, of the rigid foam. Surfactants based on polyether-modified siloxanes are particularly effective and therefore represent the preferred type of foam stabilizers.

EP1544235 describes typical polyether-modified siloxanes for rigid PU foam applications. Siloxanes having 60 to 130 silicon atoms and different polyether substituents R, the mixed molar mass of which is 450 to 1000 g/mol and the ethylene oxide content of which is 70 to 100 mol %, are used here. Although these compounds affect the fineness and regularity of the cell structure to a certain extent, there is a limit to the fine-cell content beyond which cell refinement and an associated improvement in the thermal insulation action by means of further increasing the stabilizer concentration is not possible.

Besides this, the prior art also envisages other options for positively influencing the thermal insulation capacity of the rigid foam. For instance, EP0551636A1 describes a process for producing rigid polyurethane foams by reacting polyisocyanates with at least one relatively high molecular weight compound having at least two reactive hydrogen atoms in the presence of blowing agents and catalysts, the blowing agent used being a mixture comprising 5% to 40% by weight of at least one highly fluorinated and/or perfluorinated organic compound and 30% to 95% by weight of cyclopentane. According to the statements made therein, this process is intended to enable lower thermal conductivity.

EP1553129A1 describes a process for producing a rigid polyurethane foam, comprising the production of a polyol mixture from polyol and a nucleating agent and the reaction of the polyol mixture with polyisocyanate, the production of the polyol mixture comprising emulsification of the nucleating agent with a portion or the entirety of the polyol. The nucleating agent may comprise a perfluorinated alkene containing at least 6 carbons. According to the statements made therein, this process is intended to enable improved thermal insulation properties.

However, there remains a need for options for enabling the provision of polyurethane foams having improved thermal insulation action. This corresponds to the object of the invention.

Surprisingly, it has now been found that the use of specific perfluoropolyethers, as will be described precisely hereafter, enables the provision of rigid polyurethane foam having improved thermal insulation properties.

Against this background, the invention provides a composition for the production of rigid polyurethane foam, comprising at least one isocyanate component, a polyol component, optionally a catalyst which catalyses the formation of a urethane or isocyanurate bond, optionally blowing agent, optionally foam stabilizer, wherein the composition additionally contains at least one perfluoropolyether, the perfluoropolyether comprising linear structures of formula (a) and/or cyclic structures of formula (b):

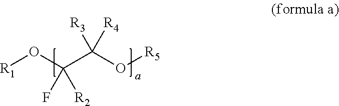

where
a=1 to 5, preferably 1 to 3,
R₁, R₅ independently of one another are —CF₃, —C₂F₅, —C₃F₇, —C₄F₉, —CF₂H, —C₂F₄H, —C₃F₆F₁ or —C₄F₈H, where the radicals having 3 or 4 carbon atoms may be linear or branched, preferably —CF₃, —C₂F₅, or —CF₂H,
R₂, R₃, R₄ independently of one another are —F or —CF₃, preferably all radicals R₂, R₃ and R₄═—F, or one of the radicals R₂, R₃, R₄ is —CF₃ and the two other radicals are —F,
with the proviso that if a>1 then different radicals R₂, R₃ and R₄, in each case according to the previous definition, are possible for each repeating unit,
and also

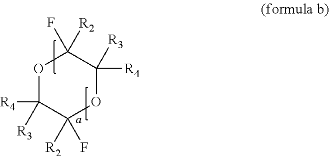

where a=1 to 4, preferably 1 to 2, particularly preferably 1 and R₂, R₃, R₄ are as defined in formula (a).

Within the context of this invention, the perfluoropolyethers are accordingly oligomeric perfluorinated ethers or perfluorinated oligomeric compounds of the corresponding alkoxides. Within the context of this invention, perfluoropolyethers according to the invention comprise at least one structure of the above formulae (a) and/or (b), especially mixtures of such structures, that is to say for example mixtures of a plurality of structures of formula (a), mixtures of a plurality of structures of formula (b) or mixtures of a plurality of structures of formulae (a) and (b).

A number of advantages accompany the invention. There is an improvement in the thermal insulation action of the resulting PU foams compared to corresponding foams without the admixture of the perfluoropolyethers to be used according to the invention. The improved thermal insulation action of the resulting PU foams was observable both in the initial state and in the aged state of the foams. All other application-relevant foam properties are only insignificantly affected, if at all, by the perfluoropolyethers to be used according to the invention. Even in the case of the quite sensitive surface quality of the foam test specimens, no changes, or at most only a marginal change, are found. Polyurethane (PU) in the context of the present invention is especially understood to mean a product obtainable by reaction of polyisocyanates and polyols or compounds having isocyanate-reactive groups. Further functional groups in addition to the polyurethane can also be formed in the reaction, examples being uretdiones, carbodiimides, isocyanurates, allophanates, biurets, ureas and/or uretonimines. Therefore, PU is understood in the context of the present invention to mean both polyurethane and polyisocyanurate, polyureas, and polyisocyanate reaction products containing uretdione, carbodiimide, allophanate, biuret and uretonimine groups. In the context of the present invention, polyurethane foam (PU foam) is especially understood to mean foam which is obtained as reaction product based on polyisocyanates and polyols or compounds having isocyanate-reactive groups. In addition to the eponymous polyurethane, further functional groups can be formed as well, examples being allophanates, biurets, ureas, carbodiimides, uretdiones, isocyanurates or uretonimines. Within the context of this invention, the term PU foams also encompasses what are called polyurethane foam mouldings, especially rigid polyurethane foam mouldings.

In a preferred embodiment of the present invention, a composition that is preferred according to the invention has the feature that the linear structures of formula (a) comprise at least 1 structure from the following group (i) to (vii):
(structure i) 1,1,1,2,3,3-hexafluoro-2,3-bis(pentafluoroethoxy)propane

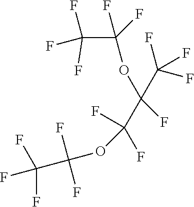 [SMILES: FC(F)(F)C(F)(F)OC(F)(F)C(F)(OC(F)(F)C(F)(F)F)C(F)(F)F]

(structure ii) 1,1,1,2,3,3-hexafluoro-3-(pentafluoroethoxy)-2-(trifluoromethoxy)propane

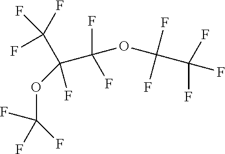 [SMILES: FC(F)(F)OC(F)(C(F)(F)F)C(F)(F)OC(F)(F)C(F)(F)F]

(structure iii) 1,1,1,2,3,3-hexafluoro-2-(pentafluoroethoxy)-3-(trifluoromethoxy)propane

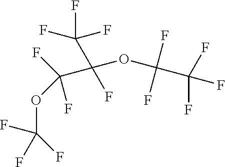 [SMILES: FC(F)(F)OC(F)(F)C(F)(OC(F)(F)C(F)(F)F)C(F)(F)F]

(structure iv) 2-(difluoromethoxy)-1,1,1,2,3,3-hexafluoro-3-(pentafluoroethoxy)propane

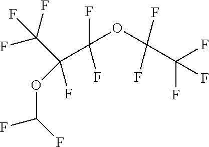 [SMILES: FC(F)OC(F)(C(F)(F)F)C(F)(F)OC(F)(F)C(F)(F)F]

(structure v) 1-(difluoromethoxy)-1,1,2,3,3,3-hexafluoro-2-(pentafluoroethoxy)propane

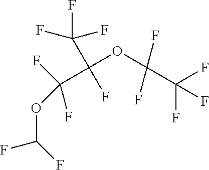 [SMILES: FC(F)OC(F)(F)C(F)(OC(F)(F)C(F)(F)F)C(F)(F)F]

(structure vi) 1,1,1,2,3,3-hexafluoro-3-{[1,1,1,2,3,3-hexafluoro-3-(trifluoromethoxy)propan yl]oxy}-2-(trifluoromethoxy)propane

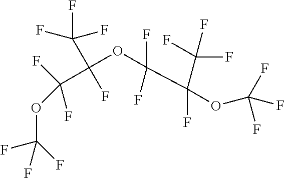 [SMILES: FC(F)(F)OC(F)(F)C(F)(OC(F)(F)C(F)(OC(F)(F)F)C(F)(F)F)C(F)(F)F]

(structure vii) 1,1,1,3,3,4,6,6,7,9,9,10,12,12,12-pentadecafluoro-4,7,10-tris(trifluoromethyl)-2,5,8,11-tetraoxadodecane

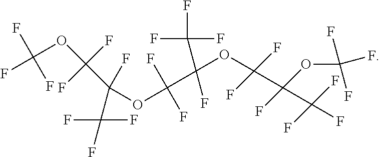 [SMILES: FC(F)(F)OC(F)(F)C(F)(OC(F)(F)C(F)(OC(F)(F)C(F)(OC(F)(F)F)C(F)(F)F)C(F)(F)F)C(F)(F)F]

Preferably, in the composition according to the invention, at least 2, more preferably at least 3, more preferably still at least 4, yet more preferably at least 5, more preferably again 6, especially all of structures (i) to (vii) are present.

Preferred combinations comprise at least the structures (i) and (ii) or at least (i) and (iii), or at least (i) and (iv), or at least (i) and (v), or at least (i) and (vi) or at least (i) and (vii), or at least (ii) and (iii) or at least (ii) and (iv), or at least (ii) and (v), or at least (ii) and (vi) or at least (ii) and (vii), or at least (iii) and (iv), or at least (iii) and (v), or at least (iii) and (vi) or at least (iii) and (vii), or at least (iv) and (v), or at least (iv) and (vi) or at least (iv) and (vii), or at least (v) and (vi) or at least (v) and (vii), or at least (vi) and (vii).

Further preferred combinations comprise at least the structures (i), (ii) and (iii), or at least (i), (ii) and (iv), or at least (i), (ii) and (v), or at least (i), (ii) and (vi), or at least (i), (ii) and (vii), or at least (i), (iii) and (iv), or at least (i), (iii) and (v), or at least (i), (iii) and (vi), or at least (i), (iii) and (vii), or at least (i), (iv) and (v), or at least (i), (iv) and (vi), or at least (i), (iv) and (vii), or at least (i), (v) and (vi), or at least (i), (v) and (vii), or at least (i), (vi) and (vii), or at least (ii), (iii) and (iv), or at least (ii), (iii) and (v), or at least (ii), (iii) and (vi), or at least (ii), (iii) and (vii), or at least (ii), (iv) and (v), or at least (ii), (iv) and (vi), or at least (ii), (iv) and (vii), or at least (iii), (iv) and (v), or at least (iii), (v) and (vi), or at least (iii), (v) and (vii), or at least (iv), (v) and (vi), or at least (iv), (v) and (vii).

In the context of a preferred embodiment, a composition according to the invention has the feature that, as cyclic structure of formula (b), at least 2,2,3,5,5,6-hexafluoro-3,6-bis(trifluoromethyl)-1,4-dioxane

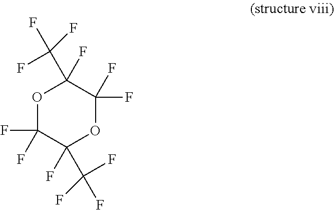 [SMILES: FC(F)(F)C1(F)OC(F)(F)C(F)(C(F)(F)F)OC1(F)F]

is present.

Particular preference is given to a composition according to the invention which contains both linear structures of formula (a) and cyclic structures of formula (b).

Preferably, the structure (viii) and additionally at least one of the structures (i) to (vii), more preferably additionally 2 or 3 of the structures (i) to (vii), are present in the composition according to the invention.

Preferred combinations comprise, in each case in addition to the structure (viii), at least also the structures (i) and (ii) or at least also (i) and (iii), or at least also (i) and (iv), or at least also (i) and (v), or at least also (i) and (vi) or at least also (i) and (vii), or at least also (ii) and (iii) or at least also (ii) and (iv), or at least also (ii) and (v), or at least also (ii) and (vi) or at least also (ii) and (vii), or at least also (iii) and (iv), or at least also (iii) and (v), or at least also (iii) and (vi) or at least also (iii) and (vii), or at least also (iv) and (v), or at least also (iv) and (vi) or at least also (iv) and (vii), or at least also (v) and (vi) or at least also (v) and (vii), or at least also (vi) and (vii).

Further preferred combinations comprise, in each case in addition to the structure (viii), at least also the structures (i), (ii) and (iii), or at least also (i), (ii) and (iv), or at least also (i), (ii) and (v), or at least also (i), (ii) and (vi), or at least also (i), (ii) and (vii), or at least also (i), (iii) and (iv), or at least also (i), (iii) and (v), or at least also (i), (iii) and (vi), or at least also (i), (iii) and (vii), or at least also (i), (iv) and (v), or at least also (i), (iv) and (vi), or at least also (i), (iv) and (vii), or at least also (i), (v) and (vi), or at least also (i), (v) and (vii), or at least also (i), (vi) and (vii), or at least also (ii), (iii) and (iv), or at least also (ii), (iii) and (v), or at least also (ii), (iii) and (vi), or at least also (ii), (iii) and (vii), or at least also (ii), (iv) and (v), or at least also (ii), (iv) and (vi), or at least also (ii), (iv) and (vii), or at least also (iii), (iv) and (v), or at least also (iii), (v) and (vi), or at least also (iii), (v) and (vii), or at least also (iv), (v) and (vi), or at least also (iv), (v) and (vii).

Further preferably, the structure (viii) and additionally at least four of the structures (i) to (vii), more preferably additionally at least 5, more preferably still additionally at least 6 of the structures (i) to (vii), in particular all of structures (i) to (vii) are present in the composition according to the invention.

Particularly preferred compositions according to the invention therefore contain all eight structures (i) to (viii).

In a particularly preferred embodiment of the invention, the composition according to the invention has the feature that the perfluoropolyethers present, taken as a whole, comprise those perfluoropolyethers as defined in formulae (a) and (b), in particular those that correspond to the structures (i) to (viii), to an extent of at least 25% by weight, preferably to an extent of at least 50% by weight, more preferably to an extent of at least 75% by weight, in particular to an extent of at least 90% by weight.

It is a particularly preferred embodiment of the invention when the perfluoropolyether used, taken as a whole, is used in a total amount of 0.01 to 15 parts, preferably 0.1 to 10 parts, particularly preferably 0.1 to 5 parts, based on 100 parts of polyols. Suitable lower limits within the scope of preferred embodiments may also be 0.3 parts, 0.5 parts or 1 part perfluoropolyether, based on 100 parts of polyols.

The perfluoropolyethers to be used according to the invention and the preparation thereof are known per se. Relevant examples can be found, inter alia, in WO2019/202079 A1, WO2019/202076 A1 or WO2018/108864 A1 and the sources cited therein.

The composition according to the invention for the production of rigid polyurethane foam can be used in all known processes to produce corresponding rigid polyurethane foam.

The present invention further provides a process for producing rigid PU foams based on foamable reaction mixtures containing polyisocyanates, compounds having reactive hydrogen atoms, blowing agents, foam stabilizers, and possibly further additives, in which perfluoropolyethers according to the invention and as described above are additionally used. Concerning the perfluoropolyethers according to the invention, reference is expressly made in this regard to the description above in order to avoid repetitions.

The invention thus further also provides a rigid PU foam produced by the above described process.

In a preferred embodiment of the invention, the rigid polyurethane foam has a foam density of 5 to 900 kg/m³, preferably 8 to 800, particularly preferably 10 to 600 kg/m³, especially 20 to 150 kg/m³.

The efficacy of the perfluoropolyethers used according to the invention is advantageously independent of the polyurethane or polyisocyanurate basic formulation, that is to say the perfluoropolyethers to be used according to the invention can be employed for improving the thermal insulation properties in a great multitude of polyurethane or polyisocyanurate formulations. A reduction in the thermal conductivity through the use of the perfluoropolyethers according to the invention can be observed both in formulations which have already been optimized with respect to low thermal conductivity exhaustively using methods known to those skilled in the art and correspond to the current prior art for use as insulating foam and also in formulations which have been optimized with respect to other foam properties and do not yet display the optimum thermal conductivity achievable by the prior art.

The rigid PU foams according to the invention have a thermal conductivity of preferably less than or equal to 25 mW/m*K, which can be markedly reduced still further by means of the optional addition of further auxiliaries and additives known to those skilled in the art. A thermal conductivity of less than 20 mW/m*K is particularly preferred.

The values for thermal conductivity of the rigid PU foams according to the invention both in the fresh and in the aged state of the foams are significantly below the thermal conductivity values of those foams that have been produced without the addition of perfluoropolyethers to be used according to the invention, but which have otherwise been produced in the same way; the thermal conductivity values are generally at least 0.3 to 1.5 mW/m*K lower.

The invention further provides for the use of the rigid PU foam according to the invention for the reduction of the energy consumption of refrigeration appliances, especially upright freezers and chest freezers, refrigerated display units and refrigerators.

The invention further provides for the use of the perfluoropolyethers according to the invention, as defined above in the description, for the
(a) production of rigid polyurethane foams, especially using a composition according to the invention,
(b) improvement of the thermal insulation properties of polyurethane foam, preferably rigid PU foam, especially in construction applications or in the refrigeration sector, and/or
(c) reduction of the thickness of a rigid PU foam insulation layer while maintaining the thermal insulation performance, especially in construction applications or in the refrigeration sector.

The perfluoropolyethers to be used according to the invention can be added directly to the reactive mixture for producing the PU foam or premixed in one of the components, preferably the blowing agent, optionally with further auxiliaries and additives. This corresponds to a preferred embodiment of the invention.

Against this background, the present invention further provides a mixture suitable for use for the production of rigid PU foams, comprising perfluoropolyethers according to the invention and as defined above in the description and at least one blowing agent selected from hydrocarbons having 3, 4 or 5 carbon atoms, preferably cyclo-, iso- and n-pentane, hydrofluorocarbons, preferably HFC 245fa, HFC 134a and HFC 365mfc, hydrochlorofluorocarbons, preferably HCFC 141b, hydrofluoroolefins (HFO) or hydrohaloolefins such as for example 1234ze, 1234yf, 1224yd, 1233zd(E) or 1336mzz, oxygen-containing compounds such as methyl formate, acetone and dimethoxymethane, or chlorinated hydrocarbons, preferably dichloromethane and 1,2-dichloroethane.

In a preferred embodiment of the invention, this mixture has the feature that the perfluoropolyether present therein, taken as a whole, comprises perfluoropolyethers according to the invention, as defined above in the description, to an extent of at least 25% by weight, preferably to an extent of at least 50% by weight, more preferably to an extent of at least 75% by weight, in particular to an extent of at least 90% by weight.

It is a further preferred embodiment of the invention when the perfluoropolyethers used, taken as a whole, are present in the mixture in a total amount of 0.1% to 50% by weight, preferably 0.5% to 30% by weight, particularly preferably 1.0% to 20% by weight.

The present invention further provides a process for producing rigid PU foam in which a mixture according to the invention and as defined above is used.

The invention further provides for the use of a mixture according to the invention and as defined above in the production of rigid PU foam, especially for improving the thermal insulation properties.

The production of rigid PU foams and the components and formulations used therefor are known per se. The perfluoropolyethers to be used according to the invention are usable in the customary foamable formulations for PU foams, in particular rigid PU foams formed from compounds having reactive hydrogen atoms (A), the polyisocyanate component (B) and customary auxiliaries and additives (C).

Polyols suitable as polyol components (A) for the purposes of the present invention are all organic substances having one or more isocyanate-reactive groups, preferably OH groups, and also formulations thereof.

Preferred polyols are all polyether polyols and/or polyester polyols and/or hydroxyl group-containing aliphatic polycarbonates, especially polyether polycarbonate polyols, and/or polyols of natural origin, known as “natural oil-based polyols” (NOPs), which are customarily used for producing polyurethane systems, such as preferably polyurethane coatings, polyurethane elastomers and especially foams. The polyols usually have a functionality of from 1.8 to 8 and preferably number-average molecular weights in the range from 500 to 15 000. The polyols having OH numbers in the range from 10 to 1200 mg KOH/g are typically used.

Isocyanates suitable as isocyanate components (B) for the purposes of this invention are all isocyanates containing at least two isocyanate groups. Generally, it is possible to use all aliphatic, cycloaliphatic, arylaliphatic and preferably aromatic polyfunctional isocyanates known per se.

Specific examples are: alkylene diisocyanates having 4 to 12 carbon atoms in the alkylene moiety, for example dodecane 1,12-diisocyanate, 2-ethyltetramethylene 1,4-diisocyanate, 2-methylpentamethylene 1,5-diisocyanate, tetramethylene 1,4-diisocyanate and preferably hexamethylene 1,6-diisocyanate (HMDI), cycloaliphatic diisocyanates such as cyclohexane 1,3- and 1,4-diisocyanate and also any mixtures of these isomers, 1-isocyanato-3,3,5-trimethyl isocyanatomethylcyclohexane (isophorone diisocyanate or IPDI for short), hexahydrotolylene 2,4- and 2,6-diisocyanate and also the corresponding isomeric mixtures, and preferably aromatic diisocyanates and polyisocyanates such as toluene 2,4- and 2,6-diisocyanate (TDI) and the corresponding isomeric mixtures, naphthalene diisocyanate, diethyltoluene diisocyanate, mixtures of diphenylmethane 2,4′- and 2,2′-diisocyanates (MDI) and polyphenyl polymethylene polyisocyanates (crude MDI) and mixtures of crude MDI and toluene diisocyanates (TDI). The organic diisocyanates and polyisocyanates can be used individually or in the form of mixtures thereof. It is likewise possible to use corresponding “oligomers” of the diisocyanates (IPDI trimer based on isocyanurate, biurets, uretdiones). In addition, the use of prepolymers based on the abovementioned isocyanates is possible.

The terms “isocyanate component” and “polyisocyanates” are used synonymously in the context of the present invention.

It is also possible to use isocyanates which have been modified by the incorporation of urethane, uretdione, isocyanurate, allophanate and other groups, called modified isocyanates.

Particularly suitable organic polyisocyanates which are therefore used with particular preference are various isomers of toluene diisocyanate (toluene 2,4- and 2,6-diisocyanate (TDI), in pure form or as isomer mixtures of various composition), diphenylmethane 4,4′-diisocyanate (MDI), “crude MDI” or “polymeric MDI” (contains the 4,4′ isomer and also the 2,4′ and 2,2′ isomers of MDI and products having more than two rings) and also the two-ring product which is referred to as “pure MDI” and is composed predominantly of 2,4′ and 4,4′ isomer mixtures, and prepolymers derived therefrom. Examples of particularly suitable isocyanates are detailed, for example, in EP 1712578, EP 1161474, WO 00/58383, US 2007/0072951, EP 1678232 and WO 2005/085310, which are hereby fully incorporated by reference.

A preferred ratio of isocyanate and polyol, expressed as the index of the formulation, that is to say as stoichiometric ratio of isocyanate groups to isocyanate-reactive groups (e.g. OH groups, NH groups) multiplied by 100, is in the range from 10 to 1000, preferably 80 to 500. An index of 100 represents a molar reactive group ratio of 1:1.

The auxiliaries and additives (C) used may in particular be the compounds that are customary for the formulation of PU foams, in particular rigid PU foams, including catalysts, foam stabilizers, blowing agents, flame retardants, fillers, colourants and light stabilizers.

Suitable catalysts for the purposes of the present invention are substances catalysing the gel reaction (isocyanate-polyol), the blowing reaction (isocyanate-water) or the di- or trimerization of the isocyanate. It is possible here to make use of the customary catalysts known from the prior art, including, for example, amines (cyclic, acyclic; monoamines, diamines, oligomers having one or more amino groups), ammonium compounds, organometallic compounds and metal salts, preferably those of tin, iron, bismuth, potassium and zinc. In particular, it is possible to use mixtures of a plurality of components as catalysts. Suitable amounts used depend on the type of catalyst and are in particular in the range from 0.05 to 5 parts by weight, or 0.1 to 10 parts by weight for potassium salts, based on 100 parts by weight of polyol.

Suitable foam stabilizers are surface-active substances such as for example organic surfactants or preferably polyether-modified siloxanes (PES). In the context of this invention, it is possible here to use any of those that facilitate foam production (stabilization, cell regulation, cell opening, etc.). These compounds are sufficiently well known from the prior art. Typical amounts of polyether siloxane foam stabilizers used are preferably from 0.5 to 5 parts by weight per 100 parts by weight of polyol, preferably from 1 to 3 parts by weight per 100 parts by weight of polyol.

Corresponding PES usable in the context of this invention are described, for example, in the following patent specifications: CN 103665385, CN 103657518, CN 103055759, CN 103044687, US 2008/0125503, US 2015/0057384, EP 1520870 A1, EP 1211279, EP 0867464, EP 0867465, EP 0 0275563.

Water is preferably added as chemical blowing agent to the foamable formulation, since it reacts with isocyanates with the evolution of carbon dioxide gas. Suitable water contents for the purposes of this invention depend on whether or not physical blowing agents are used in addition to the water. In the case of purely water-blown foams, the values are preferably in the range from 1 to 20 parts by weight per 100 parts by weight of polyol, but, when other blowing agents are used in addition, the amount used is preferably reduced to 0.1 to 5 parts by weight per 100 parts by weight of polyol.

Physical blowing agents used may be corresponding compounds having appropriate boiling points. It is likewise possible to use chemical blowing agents which react with NCO groups to liberate gases, for example the already mentioned water or formic acid. Examples of blowing agents are liquefied CO2, nitrogen, air, volatile liquids, for example hydrocarbons having 3, 4 or 5 carbon atoms, preferably cyclo-, iso- and n-pentane, hydrofluorocarbons, preferably HFC 245fa, HFC 134a and HFC 365mfc, hydrochlorofluorocarbons, preferably HCFC 141b, hydrofluoroolefins (HFO) or hydrohaloolefins such as for example 1234ze, 1234yf, 1224yd, 1233zd(E) or 1336mzz, oxygen-containing compounds such as methyl formate, acetone and dimethoxymethane, or chlorinated hydrocarbons, preferably dichloromethane and 1,2-dichloroethane.

As additives, it is possible to use all substances which are known from the prior art and are used in the production of polyurethanes, especially polyurethane foams, for example crosslinkers and chain extenders, stabilizers against oxidative degradation (known as antioxidants), flame retardants, surfactants, biocides, cell openers, solid fillers, antistatic additives, thickeners, dyes, pigments, colour pastes, fragrances, emulsifiers etc.

Insulation foams for the thermal insulation of buildings are subject to fire safety requirements. Flame retardants usable for this purpose are preferably liquid organophosphorus compounds such as halogen-free organophosphates, e.g. triethyl phosphate (TEP), halogenated phosphates, for example tris(1-chloro-2-propyl) phosphate (TCPP) and tris(2-chloroethyl) phosphate (TCEP), and organic phosphonates, for example dimethyl methanephosphonate (DMMP), dimethyl propanephosphonate (DMPP), or solids such as ammonium polyphosphate (APP) and red phosphorus. Suitable flame retardants further include halogenated compounds, for example halogenated polyols, and also solids such as expandable graphite and melamine.

The present invention additionally provides for the use of polyurethane foams according to the invention as insulating panels and insulant, and also a cooling apparatus which includes a polyurethane foam according to the invention as insulating material.

The invention yet further provides for the use of the perfluoropolyethers according to the invention, as characterized hereinabove in the description, for the reduction of the thickness of a rigid PU foam insulation layer while maintaining the thermal insulation performance, especially in insulating panels and insulants.

A preferred PU foam formulation in the context of this invention comprises the perfluoropolyethers according to the invention and results in a foam density of 10 to 900 kg/m³ and has the following composition, according to a preferred embodiment of the invention:

| | Proportion |
|---|---|
| Component | by weight |
| Polyol | 0.1 to 100 |
| Amine catalyst | 0 to 5 |
| Metal catalyst | 0 to 10 |
| Polyether siloxane | 0.1 to 5 |
| Water | 0.01 to 20 |
| Blowing agent | 0 to 40 |
| Perfluoropolyethers according to the invention | >0 to 5 |
| Further additives (flame retardants, etc.) | 0 to 90 |
| Isocyanate index: 10 to 1000 | |

The formulations according to the invention can be processed to give the desired PU foams by any methods familiar to those skilled in the art.

Rigid polyurethane foam or rigid PU foam is an established technical term. The known and fundamental difference between flexible foam and rigid foam is that flexible foam shows elastic characteristics and hence deformation is reversible. By contrast, rigid foam is permanently deformed. In the context of the present invention, rigid polyurethane foam is especially understood to mean a foam to DIN 7726 that has a compressive strength to DIN 53 421/DIN EN ISO 604:2003-12 of advantageously 20 kPa, by preference 80 kPa, preferably ≥100 kPa, more preferably 150 kPa, particularly preferably 180 kPa. In addition, the rigid polyurethane foam, according to DIN EN ISO 4590:2016-12, advantageously has a closed-cell content of greater than 50%, preferably greater than 80% and particularly preferably greater than 90%.

The rigid PU foams according to the invention can be used as or for production of insulation materials, preferably insulating panels, refrigerators, insulating foams, roof liners, packaging foams or spray foams.

The PU foams according to the invention can be used advantageously particularly in the refrigerated warehouse, refrigeration appliances and domestic appliances industry, for example for production of insulating panels for roofs and walls, as insulating material in containers and warehouses for frozen goods, and for refrigeration and freezing appliances.

Further preferred fields of use are in vehicle construction, especially for production of vehicle inner roof liners, bodywork parts, interior trim, cooled vehicles, large containers, transport pallets, packaging laminates, in the furniture industry, for example for furniture parts, doors, linings, in electronics applications.

The invention further provides for the use of the rigid PU foam as insulation material in refrigeration technology, in refrigeration equipment, in the construction sector, automobile sector, shipbuilding sector and/or electronics sector, as insulating panels, as spray foam or as one-component foam.

The examples which follow describe the present invention by way of example, without any intention of restricting the invention, the scope of which is apparent from the entirety of the description and the claims, to the embodiments cited in the examples.

### EXAMPLES

### Example 1: Rigid PU Foam

The following foam formulation was used for the performance comparison:

| | Proportion |
|---|---|
| Component | by weight |
| Polyol* | 97.0 |
| POLYCAT ® 5** | 0.5 |
| POLYCAT ® 8** | 1.0 |
| POLYCAT ® 41** | 0.4 |
| DABCO ® TMR 2** | 0.8 |
| BDMA** | 1.2 |
| Polyether siloxane*** | 3 |
| HFC-245fa | 5.5 |
| Cyclopentane | 11.5 |
| Perfluoropolyethers according to the invention**** | 2.0 |
| MDI***** | 140.4 |

*Polyether polyol based on sucrose, sorbitol, o-TDA and glycerol
**Catalysts from Evonik Industries AG
***TEGOSTAB ® B 84813 from Evonik Industries AG
****Perfluoropolyethers having structures (i), (ii) and (iii)
*****Polymeric MDI, 200 mPa*s, 31.5% NCO, functionality 2.7.

The comparative foamings were carried out by hand mixing. For this purpose, polyol, catalysts, water, foam stabilizer, perfluoropolyether and blowing agent were weighed into a beaker and mixed by means of a disc stirrer (diameter 6 cm) at 1000 rpm for 30 s. The blowing agent quantity which had evaporated during the mixing operation was determined by reweighing and replenished. The MDI was now added, the reaction mixture was stirred with the stirrer described at 3000 rpm for 7 s and immediately transferred into an aluminium mould thermostatted to 45° C. and having a size of 145 cm×14 cm×3.5 cm, the mould being inclined at an angle of 10° (along the 145 cm long side) and lined with polyethylene film. The foam formulation was in this case introduced at the lower side, so that the expanding foam fills the mould in the feed region and rises in the direction of the higher side. The amount of foam formulation used was calculated such that it was 10% above the amount necessary for minimum filling of the mould.

After 10 min, the foams were demoulded. One day after foaming, the foams were analysed. Surface and internal defects were assessed subjectively on a scale from 1 to 10, where 10 represents an (idealized) impeccable foam and 1 represents a very significantly defective foam. The thermal conductivity coefficient (A value in mW/m·K) was measured on 2.5 cm-thick discs with a device of the Hesto Lambda Control type, model HLC X206, at an average temperature of 10° C. in accordance with the specifications of standard EN12667:2001.

The results are compiled in the table which follows:

| Parts | | |
|---|---|---|
| perfluoropolyether | | λ value in |
| in pphp (parts per | Density | mW/m · K |
| hundred polyol) | in kg/m³ | (initial) |
| 0 (reference) | 30.0 | 20.9 |
| 2.0 | 29.9 | 20.1 |

The results show that a significant improvement in the thermal conductivity can be achieved with the perfluoropolyethers according to the invention, with the values both in the fresh and in the aged state being markedly below the reference value of the foam without addition of the perfluoropolyethers.

It should be particularly emphasized here that even a very small addition of perfluoropolyethers according to the invention leads to measurable improvements.

All other application-relevant foam properties are only insignificantly affected, if at all, by the perfluoropolyethers according to the invention. Even in the case of the quite sensitive surface quality of the foam test specimens, no changes, or only a marginal deterioration, are found.

### Example 2: Rigid PIR Foam

The following foam formulation was used for the performance comparison:

| | Proportion |
|---|---|
| Component | by weight |
| Polyester polyol* | 100 |
| Amine catalyst** | 0.4 |
| Potassium trimerization catalyst*** | 5 |
| Polyether siloxane**** | 2 |
| Water | 0.8 |
| Cyclopentane/isopentane 70:30 | 19 |
| Flame retardant TCPP | 10 |
| Perfluoropolyethers according to the invention***** | 0-4 |
| MDI****** | 220 |

*Stepanpol ® PS 2412 from Stepan
**POLYCAT ® 5 from Evonik Industries AG
***KOSMOS ® 70 LO from Evonik Industries AG
****TEGOSTAB ® B 84504 from Evonik Industries AG
*****Perfluoropolyethers having structures (i), (ii) and (iii)
******Polymeric MDI, 200 mPa*s, 31.5% NCO, functionality 2.7.

The comparative foamings were carried out by hand mixing. For this purpose, polyol, catalysts, water, foam stabilizer, flame retardant, perfluoropolyether and blowing agent were weighed into a beaker and mixed by means of a disc stirrer (diameter 6 cm) at 1000 rpm for 30 s. The blowing agent quantity which had evaporated during the mixing operation was determined by reweighing and replenished. The MDI was now added, the reaction mixture was stirred with the stirrer described at 3000 rpm for 5 s and immediately transferred into an open mould having a size of 27.5×14×14 cm (W×H×D).

After 10 min, the foams were demoulded. One day after foaming, the foams were analysed. Surface and internal defects were assessed subjectively on a scale from 1 to 10, where 10 represents an (idealized) impeccable foam and 1 represents a very significantly defective foam. The thermal conductivity coefficient (A value in mW/m·K) was measured on 2.5 cm-thick discs with a device of the Hesto Lambda Control type, model HLC X206, at an average temperature of 10° C. in accordance with the specifications of standard EN12667:2001. For the determination of an ageing value for the thermal conductivity, the test specimens were stored at 70° C. over 7 days and then measured again.

The results are compiled in the table which follows:

| Parts | | | |
|---|---|---|---|
| perfluoropolyether | | λ value in | λ value in |
| in pphp (parts per | Density | mW/m · K | mW/m · K |
| hundred polyol) | in kg/m³ | (initial) | (aged) |
| 0 (reference) | 31.1 | 24.0 | 27.7 |
| 0.1 | 31.5 | 23.5 | 27.2 |
| 0.3 | 31.4 | 23.5 | 27.3 |
| 0.5 | 31.1 | 23.4 | 27.3 |
| 1.0 | 30.7 | 22.9 | 26.8 |
| 4.0 | 30.5 | 22.7 | 26.6 |

The results again show that a significant improvement in the thermal conductivity can be achieved with the perfluoropolyethers according to the invention, with the values both in the fresh and in the aged state here too being markedly below the reference value of the foam without addition of the perfluoropolyethers.

It should be particularly emphasized here that even a very small addition of perfluoropolyethers according to the invention leads to measurable improvements.

All other application-relevant foam properties are only insignificantly affected, if at all, by the perfluoropolyethers according to the invention. Even in the case of the quite sensitive surface quality of the foam test specimens, no changes, or only a marginal deterioration, are found.

### Example 3: Rigid PIR Foam

The following foam formulation was used for the performance comparison:

| | Proportion |
|---|---|
| Component | by weight |
| Polyester polyol* | 100 |
| Amine catalyst** | 0.4 |
| Potassium trimerization catalyst*** | 5 |
| Polyether siloxane**** | 2 |
| Water | 0.8 |
| Cyclopentane/isopentane 70:30 | 19 |
| Flame retardant TCPP | 10 |
| Perfluoropolyethers according to the invention***** | 0.1-4 |
| MDI****** | 220 |

*Stepanpol ® PS 2412 from Stepan
**POLYCAT ® 5 from Evonik Industries AG
***KOSMOS ® 70 LO from Evonik Industries AG
****TEGOSTAB ® B 84504 from Evonik Industries AG
*****Perfluoropolyethers having structures (i), (ii) and (iii)
******Polymeric MDI, 200 mPa*s, 31.5% NCO, functionality 2.7.

The comparative foamings were carried out by hand mixing. For this purpose, polyol, catalysts, water, foam stabilizer, flame retardant, perfluoropolyether and blowing agent were weighed into a beaker and mixed by means of a disc stirrer (diameter 6 cm) at 1000 rpm for 30 s. The blowing agent quantity which had evaporated during the mixing operation was determined by reweighing and replenished. The MDI was now added, the reaction mixture was stirred with the stirrer described at 3000 rpm for 5 s and immediately transferred into an aluminium mould thermostatted to 60° C. and having a size of 25 cm×50 cm×7 cm, the mould being lined with polyethylene film.

After 10 min, the foams were demoulded. One day after foaming, the foams were analysed. Surface and internal defects were assessed subjectively on a scale from 1 to 10, where 10 represents an (idealized) impeccable foam and 1 represents a very significantly defective foam. The thermal conductivity coefficient (λ value in mW/m·K) was measured on 2.5 cm-thick discs with a device of the Hesto Lambda Control type, model HLC X206, at an average temperature of 10° C. in accordance with the specifications of standard EN12667:2001. For the determination of an ageing value for the thermal conductivity, the test specimens were stored at 70° C. over 7 days and then measured again.

The results are compiled in the table which follows:

| Parts | | | |
|---|---|---|---|
| perfluoropolyether | | λ value in | λ value in |
| in pphp (parts per | Density | mW/m · K | mW/m · K |
| hundred polyol) | in kg/m³ | (initial) | (aged) |
| 0 (reference) | 34.1 | 21.4 | 24.0 |
| 1.0 | 33.2 | 20.6 | 23.5 |
| 4.0 | 33.2 | 20.2 | 22.9 |

The results again show that a significant improvement in the thermal conductivity can be achieved with the perfluoropolyethers according to the invention, with the values both in the fresh and in the aged state here too being markedly below the reference value of the foam without addition of the perfluoropolyethers.

It should be particularly emphasized here that even a very small addition of perfluoropolyethers according to the invention leads to measurable improvements.

All other application-relevant foam properties are only insignificantly affected, if at all, by the perfluoropolyethers according to the invention. Even in the case of the quite sensitive surface quality of the foam test specimens, no changes, or only a marginal deterioration, are found.

1-14. (canceled)

15. A composition for the production of rigid polyurethane foam, comprising an isocyanate component, a polyol component, and at least one perfluoropolyether comprising a linear structure of formula (a): 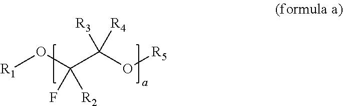
wherein for formula (a):
a=1 to 5;
R₁ and R₅ are, independently of one another, —CF₃, —C₂F₅, —C₃F₇, —C₄F₉, —CF₂H, —C₂F₄H,
—C₃F₆H or C₄F₈H, wherein the radicals having 3 or 4 carbon atoms may be linear or branched;
R₂, R₃, R₄ independently of one another are —F or —CF₃;
and/or a cyclic structure of formula (b): 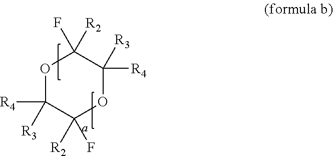
wherein for formula (b), a=1 to 4, and R₂, R₃, R₄ are as defined in formula (a);
and wherein the composition optionally also comprises: a catalyst that catayses the formation of a urethane or isocyanurate bond; a blowing agent; and/or a foam stabilizer.

16. The composition of claim 15, wherein R₁ and R₅ are independently of one another —CF₃, —C₂F₅, or —CF₂H.

17. The composition of claim 15, wherein, when a=1, all radicals R₂, R₃ and R₄═—F, or one of the radicals R₂, R₃, R₄ is —CF₃ and the two other radicals are —F.

18. The composition of claim 15, wherein for formula (b), a=1.

19. The composition of claim 15, wherein for formula (b), a=2.

20. The composition of claim 16, wherein, when a=1, all radicals R₂, R₃ and R₄═—F, or one of the radicals R₂, R₃, R₄ is —CF₃ and the two other radicals are —F.

21. The composition of claim 20, wherein for formula (b), a=1.

22. The composition of claim 20, wherein for formula (b), a=2.

23. The composition of claim 15, wherein the linear structures of formula (a) comprise at least one structure selected from group (i) to (vii) as follows:
(structure i): 1,1,1,2,3,3-hexafluoro-2,3-bis(pentafluoroethoxy)propane: 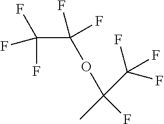
(structure ii): 1,1,1,2,3,3-hexafluoro-3-(pentafluoroethoxy)-2-(trifluoromethoxy)propane: 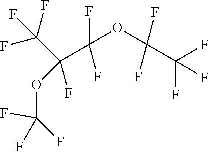 [SMILES: FC(F)(F)OC(F)(C(F)(F)F)C(F)(F)OC(F)(F)C(F)(F)F]
(structure iii): 1,1,1,2,3,3-hexafluoro-2-(pentafluoroethoxy)-3-(trifluoromethoxy)propane: 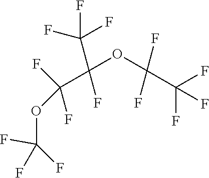 [SMILES: FC(F)(F)OC(F)(F)C(F)(OC(F)(F)C(F)(F)F)C(F)(F)F]
(structure iv): 2-(difluoromethoxy)-1,1,1,2,3,3-hexafluoro-3-(pentafluoroethoxy)propane: 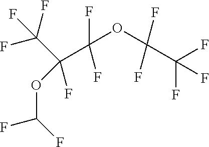 [SMILES: FC(F)OC(F)(C(F)(F)F)C(F)(F)OC(F)(F)C(F)(F)F]
(structure v): 1-(difluoromethoxy)-1,1,2,3,3,3-hexafluoro-2-(pentafluoroethoxy)propane: 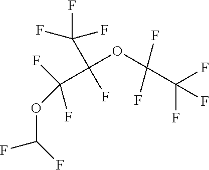 [SMILES: FC(F)OC(F)(F)C(F)(OC(F)(F)C(F)(F)F)C(F)(F)F]
(structure vi): 1,1,1,2,3,3-hexafluoro-3-{[1,1,1,2,3,3-hexafluoro (trifluoromethoxy)propan-2-yl]oxy}-2-(trifluoromethoxy)propane: 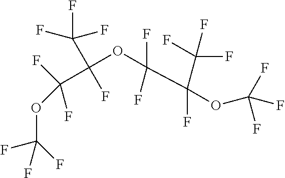 [SMILES: FC(F)(F)OC(F)(F)C(F)(OC(F)(F)C(F)(OC(F)(F)F)C(F)(F)F)C(F)(F)F]
(structure vii): 1,1,1,3,3,4,6,6,7,9,9,10,12,12,12-pentadecafluoro-4,7,10-tris(trifluoro methyl)-2,5,8,11-tetraoxadodecane:

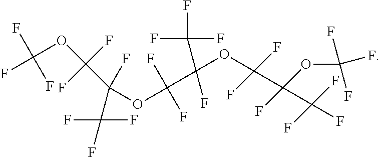 [SMILES: FC(F)(F)OC(F)(F)C(F)(OC(F)(F)C(F)(OC(F)(F)C(F)(OC(F)(F)F)C(F)(F)F)C(F)(F)F)C(F)(F)F]

24. The composition of claim 23, wherein the linear structures of formula (a) comprise at least two structures selected from groups (i) to (vii).

25. The composition of claim 23, wherein the linear structures of formula (a) comprise at least six structures selected from groups (i) to (vii).

26. The composition of claim 15, comprising as a cyclic structure of formula (b), 2,2,3,5,5,6-hexafluoro-3,6-bis(trifluoromethyl)-1,4-dioxane:

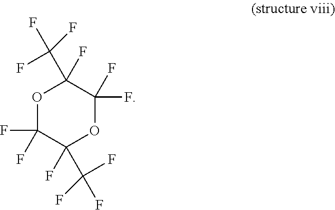 [SMILES: FC(F)(F)C1(F)OC(F)(F)C(F)(C(F)(F)F)OC1(F)F]

27. The composition of claim 15, wherein both linear structures of formula (a) and cyclic structures of formula (b) are present.

28. The composition of claim 15, wherein the perfluoropolyethers comprise at least 25% by weight of the total perfluoropolyethers present.

29. The composition of claim 15, wherein the perfluoropolyethers used, are present in a total amount of 0.01 to 15 parts per 100 parts of polyol.

30. A process for producing PU foams, based on foamable reaction mixtures containing polyisocyanates, compounds having reactive hydrogen atoms, blowing agents, foam stabilizers, and optionally further additives, wherein the perfluoropolyethers of claim 15 are additionally used.

31. A mixture suitable for the production of a rigid PU foam, comprising the perfluoropolyethers of claim 15 and at least one blowing agent selected from a hydrocarbon having 3, 4 or 5 carbon atoms.

32. The mixture of claim 31, wherein the hydrocarbon is an oxygen-containing hydrocarbon or a chlorinated hydrocarbon.

33. The mixture of claim 31, comprising at least 26% by weight of the perfluoropolyethers.

34. The mixture of claim 31, wherein the perfluoropolyethers, taken as a whole, are present in a total amount of 0.1% to 50% by weight.
